(19) United States
(12) Patent Application Publication
(10) Pub. No.: US 2019/0099346 A1
(43) Pub. Date: Apr. 4, 2019

(54) Keratin Treatments

(71) Applicant: Revlon Consumer Products Corporation, New York, NY (US)

(72) Inventors: David Valia, San Diego, CA (US); Mark Kaiser, Midland, MI (US); Chad Conger, San Marcos, CA (US); Sebastian Emery Scheibert, Sanford, MI (US)

(21) Appl. No.: 15/721,988

(22) Filed: Oct. 2, 2017

(51) Int. Cl. A61K 8/65; A61K 8/88; A61K 8/73; A61K 8/37; A61Q 3/00; A61Q 5/00; A61Q 19/00

(52) U.S. Cl. CPC A61K 8/65; A61K 8/88; A61K 8/731; A61K 8/37; A61K 2800/59; A61Q 5/00; A61Q 19/00; A61K 2800/544; A61K 2800/43; A61Q 3/00

(57) ABSTRACT

The present disclosure relates generally to keratin treatments. More particularly, but not by way of limitation, present disclosure relates to skin, nail, hair, and eyelash treatments comprising a keratin-peptide/dendrimer composition for ameliorating a condition of a nail. The keratin-peptide can be keratin or hydrolyzed or partially hydrolyzed keratin.

### FIELD

The present disclosure relates generally to keratin treatments. More particularly, but not by way of limitation, present disclosure relates to skin, nail, hair, and eyelash treatments comprising a keratin-peptide/dendrimer composition for ameliorating a condition of the skin, nail, hair or eyelash.

### BACKGROUND

The information provided below is not admitted to be prior art to the present invention, but is provided solely to assist the understanding of the reader.

Keratin is a term used to describe various structural proteins found in parts of living animals, particularly in mammals and humans. For example, keratin is present in the outer layers of the skin and is a major component of hair, including eyelashes, and nails, including fingernails and toenails. In non-human animals, keratin is a major component of horns, claws, hooves, shells, feathers, and beaks.

In humans, hair, nails and other keratin containing structures in humans can become brittle or cracked and can become subject to breaking, resulting in a loss of attractiveness. One way in which this loss of attractiveness is alleviated is by covering the imperfections, for example with nail polish. Alternatively, temporary products, such as hair conditioners, can be applied to hide or mask the condition. There are some available treatments for improving such conditions, for example the application of hydrolyzed keratin. However, such treatments are exceedingly temporary, often removed during hand or hair washing or even simply rinsing.

There remains a need for treatment of keratin containing structures that is effective and long lasting.

### SUMMARY

In order to meet this need, the present disclosure provides keratin restorative compositions. Keratin restorative compositions of the invention include a keratin-peptide and a dendrimer. The weight ratio of keratin-peptide to dendrimer can be from about 4:1 to about 1:60. The keratine-peptide can include a partially-hydrolyzed keratin. The dendrimer can include one or more functional groups that can associate, interact, or react with one or more functional groups on the keratin-peptide. For example, the one or more functional groups on the dendrimer may be selected from OH, NH₂, SH, CO₂H or others. The keratin-peptide and the dendrimer can be associated by the formation of a covalent, ionic or hydrogen bond or through other intermolecular interactions such as van der Waals forces, dipole-dipole interactions and the like. In some embodiments, the keratin-peptide remains at least partially exposed for interaction or reaction with keratin of a keratinaceous substrate and the dendrimer functional group extends unidirectionally from the keratin-peptide constituent. Exemplary schematic formulas for the keratin-peptide/dendrimer composition and dendrimer are shown in FIG. 1 and FIG. 2, respectively. Dendrimers used in the keratin restorative composition of the invention can include at least one hydrophobic group. The hydrophobic group can include, a C₆-C₂₀ linear or branched hydrocaebon chain, optionally substituted by one or more functional groups.

Keratin restorative compositions according to the invention can further include a solvent. Additional components in the keratin restorative composition can include one or more of a film forming polymer, a reactive (meth)acrylate, an epoxidized esterified plant oil, a polyhedral oligomeric silsesquioxane, and a colorant. The film forming polymer can be, for example, a cellulose ester, nitrocellulose, or a cellulose acetate alkylate.

The invention includes a method for ameliorating a condition of a keratinaceous substrate by applying to the keratinaceous substrate a keratin restorative composition of the invention. The keratinaceous substrate can be skin, nails and hair. Conditions to be ameliorated can be nail delamination, brittleness, cracking, chipping, splitting, dryness, and texturing.

Keratin restorative compositions of the invention can be made by combining a keratin-peptide and a dendrimer. Combining can include chemically reacting by forming a covalent, ionic, or hydrogen bond. Combining can also include mixing of the keratin-peptide and the dendrimer. The bond between the keratin-peptide and the dendrimer can be formed by the association of the reactive functional groups of the keratin-peptide and the dendrimer.

Further objectives and advantages, as well as the structure and function of preferred embodiments will become apparent from a consideration of the description, drawings, and examples.

### BRIEF DESCRIPTION OF THE FIGURES

The foregoing and other features and advantages of the invention will be apparent from the following, more particular description of embodiments of the invention, as illustrated in the accompanying drawings wherein;

FIG. 1 is a schematic structure of a keratin-peptide/dendrimer composition useful in compositions according to the invention; and

FIG. 2 is a schematic representation of a dendrimer unit useful in preparing compositions of the present invention.

### DESCRIPTION

Embodiments of the present invention are discussed in detail below. In describing embodiments, specific terminology is employed for the sake of clarity. However, the invention is not intended to be limited to the specific terminology so selected. While specific exemplary embodiments are discussed, it should be understood that this is done for illustration purposes only. A person skilled in the relevant art will recognize that other components and configurations can be used without parting from the spirit and scope of the invention. While a number of embodiments and features are described herein, it is to be understood that the various features of the invention and aspects of embodiments, even if described separately, may be combined unless mutually exclusive or contrary to the specific description. All references cited herein are incorporated by reference as if each had been individually incorporated.

Hydrolyzed keratin has been used as a cosmetic treatment for keratinaceous materials such as skin. One of the drawbacks of the use of hydrolyzed keratin is its water solubility. Although hydrolyzed keratin may interact with keratin in, for example, skin, hair or nails, it remains a water soluble compound that can be released and rinsed away by water or other aqueous systems. The same drawback may be observed with the use of other keratin-derived materials. The present invention is directed to treatment compositions and restorative compositions that overcome this and other drawbacks associated with the use of existing hydrolyzed keratin treatments.

The present invention provides a treatment composition or formulation, methods of making the treatment composition or formulation, and methods of using the treatment composition or formulation. The terms “treatment”, “restorative treatment”, “keratin treatment”, “restorative keratin treatment”, and the like are used interchangeably throughout this specification. These terms refer to compositions and formulations applied to keratinaceous substrates to prevent, treat, or ameliorate delamination, dryness, brittleness, cracking, chipping, splitting and other natural degradation of keratin material or the keratinaceous substrate. The composition and formulation can also be applied to a keratinaceous substrate to improve or enhance the appearance, attractiveness or texture.

Treatment compositions according to the invention include a combination of a keratin-peptide and a dendrimer. The keratin-peptide and dendrimer can be combined to form a keratin-peptide/dendrimer composition in which the keratin-peptide and the dendrimer are associated, as described further below. For example, the keratin-peptide/dendrimer composition can include a keratin-peptide with a dendrimer unit attached thereto.

Keratin-Peptide.

As used in the present disclosure, the term keratin-peptide refers to a keratin, a peptide derived from a keratin, a peptide that is structurally similar to keratin or structurally similar to a peptide derived from keratin. For example, keratin-peptide can refer to a peptide having a high cystine content. An exemplary keratin-peptide is hydrolyzed keratin. In an embodiment, at least a portion of the cystines present in the keratin-peptide are in an S-sulphonated form. A non-limiting, suitable keratin-peptide is commercially-available as ProSina™ (Keratec Ltd., Canterbury, NZ), which is described as a purified protein faction isolated from wool which leaves the natural cystine content of the keratin in an active, S-sulpho, form.

According to an aspect, the present invention provides a composition having a dendrimer and a keratin-peptide for bonding to or coating a keratinaceous substrate to prevent or restore delamination, dryness, brittleness, breaking or cracking, ridges, splitting, and texturing or other degradations or imperfections in the keratinaceous substrate. As used herein, a keratinaceous substrate is a material containing or composed of keratin. Examples of keratinaceous substrates include, for example, skin, hair (including eyelashes), and nails (including toenails and fingernails). According to an aspect, the keratin-peptide includes a plurality of cystine units present in an S-sulphonated form. According to a further aspect, the keratin-peptide includes a partially-hydrolyzed keratin. Exemplary keratin-peptides can be extracted from keratinaceous substances. For example, a suitable keratin-peptide is a keratin extracted from wool and sold under the trademark ProSina™.

Without being bound by theory, it is believed that the keratin-peptide used in treatment compositions of the present invention interacts with keratin in the keratinaceous substrate upon application of the treatment composition. This interaction may be by, for example, formation of bonds between the keratin in the keratinaceous substrate and the keratin-peptide in the treatment composition, or physically coating the keratinaceous substrate. As used herein, the term “bond” is to be interpreted in the broadest sense to include intermolecular interactions that cause association between the keratin in the keratinaceous substrate and the keratin-peptide in the treatment composition. For example, as used herein the term bond includes van der Waal's interactions, as well as covalent, ionic, and hydrogen bonds. The interaction between the keratin substrate and keratin-peptide may further result in cross-linking or other reinforcement of the keratinaceous substrate to provide physical and structural support and enhancement. Alternatively, the composition of the present invention may provide a physical coating of the keratin-peptide/dendrimer composition on the keratinaceous substrate.

Dendrimers.

Dendrimers are repetitively branched molecules. Typically, dendrimers are associated symmetrically about a core and can assume a three-dimensional structure. Dendrimers can be prepared from individual dendrimer units where a first dendrimer unit is attached to a core or base molecule and two (or more) second dendrimer units are attached to the first dendrimer unit. This process is repeated to add two dendrimer units to each of the second dendrimer units until the desired size and branching of the overall structure is achieved.

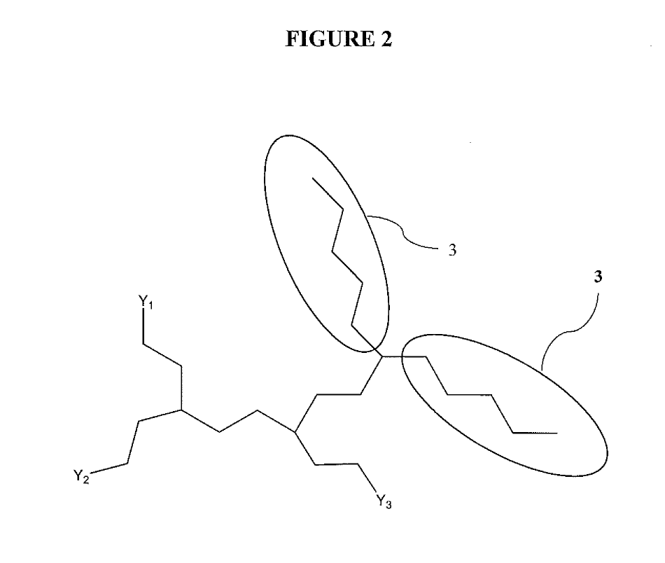

In some embodiments, the dendrimer component may contain hydrophobic groups. Such a structure is shown schematically in FIG. 2 and includes a hydrophobic group 3 on the dendrimer unit. The groups Y₁, Y₂, and Y₃ are functional groups used to interact, associate or react with a complementary functional group on the keratin-peptide, with further dendrimer units to increase branching, or with additional hydrophobic groups. For example, Y₁, Y₂, and Y₃ can independently be OH, NH₂, SH or COOH, as well as others. When a hydrophobic group R′ is present, Y₁, Y₂, and Y₃ can independently be, for example, OH, NH₂, SH, COOH, OR′, NHR′, SR′H, COOR′ or NR′₂. The hydrophobic group R′ can include, for example, a linear or branched C₆-C₂₀ carbon chain which can have additional functional groups present so long as the additional functional groups do not adversely affect the hydrophobic nature of the hydrophobic portion of the dendrimer unit. In the schematic of FIG. 2, two hydrophobic groups 3 are illustrated. However, the dendrimer, or individual dendrimers within a mixture of dendrimers, may have no hydrophobic groups, one hydrophobic group, two hydrophobic groups or three or more hydrophobic groups. Further, the bond vertices illustrated in FIG. 2 are not intended to be limited to C, but may also be N, S, O or any other suitable atom. The dendrimer unit may also contain other functional groups such as carboxyl groups and the like.

Suitable dendrimers can be prepared through modification of existing dendrimer units by incorporating a hydrophobic unit to provide a hydrophobic portion to the molecule. Alternatively, a hydrophobic moiety can be attached to the dendrimer portion of a keratin-peptide/dendrimer composition after combining the dendrimer and the keratin-peptide. The hydrophobic portion may include, for example, a linear or branched C₆-C₂₀ carbon chain which can have additional functional groups present so long as the additional functional groups do not adversely affect the hydrophobic nature of the hydrophobic portion of the dendrimer unit. As will be appreciated by persons skilled in the art, a number of smaller, less hydrophobic groups can be placed on one or more dendrimer units in order to provide a structure with hydrophobic portions and/or overall hydrophobicity in a part of the dendrimer or the dendrimer as a whole. In some embodiments, the carbon chain can be derived from a fatty acid and attached to the dendrimer via formation of an ester or amide linkage. In other embodiments, a suitably functionalized dendrimer unit can be reacted with a 1-alkene in an addition reaction. In still other embodiments, a 1-alkene can be epoxidized and reacted with a suitably functionalized dendrimer unit via a ring opening reaction resulting in a 2-hydroxy substituent on the hydrophobic portion of the dendrimer unit. In performing such reactions or others, it will be appreciated that one, two, three, four or more hydrophobic groups may be incorporated into a dendrimer. Persons skilled in the art will recognize many other coupling reactions that can be used to incorporate a hydrophobic group onto the dendrimer unit or a preformed keratin-peptide/dendrimer composition.

In an exemplary embodiment, the dendrimer unit can have the structure shown in Formula I, wherein R may be a functional group such as OH, NH₂, SH or COOH. An exemplary dendrimer is Polyamidoamine (PAMAM) dendrimer available from Dendritech, Inc. (Midland, Mich., USA) having a structure shown in Formula I in which R═NH₂.

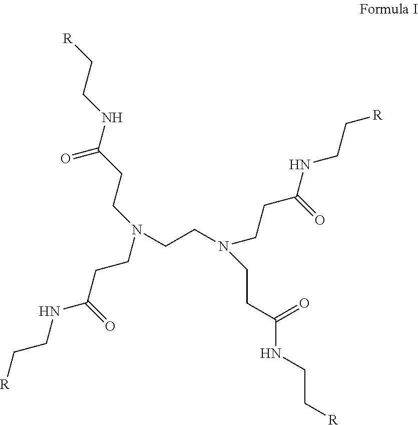

Suitable hydrophobic dendrimers may be prepared by reacting an “arm” of the dendrimer unit with one or more hydrophobic units. For example, if the dendrimer unit contains reactive hydroxyl group or amino group at the end of one or more “arms,” modification is accomplished by replacing one of the hydrogens of the pendent hydroxyl groups or amino groups with a suitable hydrophobic unit to provide a hydrophobic portion to the dendrimer unit. An exemplary structure resulting from such a modification prepared from a hydroxyl group is shown as Formula II.

In an exemplary embodiment, a hydrophobic dendrimer is prepared from dendrimer unit, for example a molecule as shown in Formula I, by reaction with a hydrophobic precursor epoxide, for example to form a structure as shown in Formula II below. The reaction product can include a mixture of compounds including the unreacted dendrimer unit, and dendrimer units having one, two, or three hydrophobic units attached. For the purposes of the invention, it is suitable to use this mixture of reaction products as the dendrimer component. In a particular embodiment, the reaction product contains about 50 mole % or less unreacted dendrimer unit, about 25% or less of dendrimer containing one hydrophobic unit, about 25% or less of dendrimer containing two hydrophobic units, and about 10 mole % or less of dendrimer containing three hydrophobic units. The product may contain approximately equal molar amounts of dendrimers containing one hydrophobic unit and dendrimers containing two hydrophobic units. As will be recognized by persons skilled in the art, the ratios and amounts of the various components in the reaction product can be controlled by adjusting the molar ratio of the dendrimer unit and hydrophobic precursor, as well as reaction conditions. Additionally, by controlling reaction conditions, it may be possible to prepare a dendrimer having two different hydrophobic groups, which could lead to a more complex mixture.

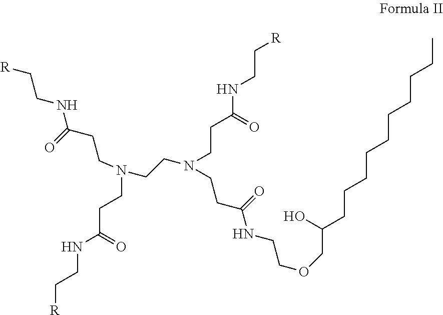

In other embodiments, the dendrimer unit may comprise reactive amino groups at the end of one or more “arms,” and the dendrimer unit may be modified by reacting with one or more hydrophobic chains at the amino end to form secondary or tertiary amine hydrophobic dendrimers. As a result, the suitable dendrimer may be a mixture of unreacted dendrimers, secondary amine containing hydrophobic dendrimer or tertiary amine containing hydrophobic dendrimer. Exemplary structures of a secondary amine containing branched hydrophobic dendrimer and a tertiary amine containing hydrophobic dendrimer are shown as Formulas III and IV, respectively.

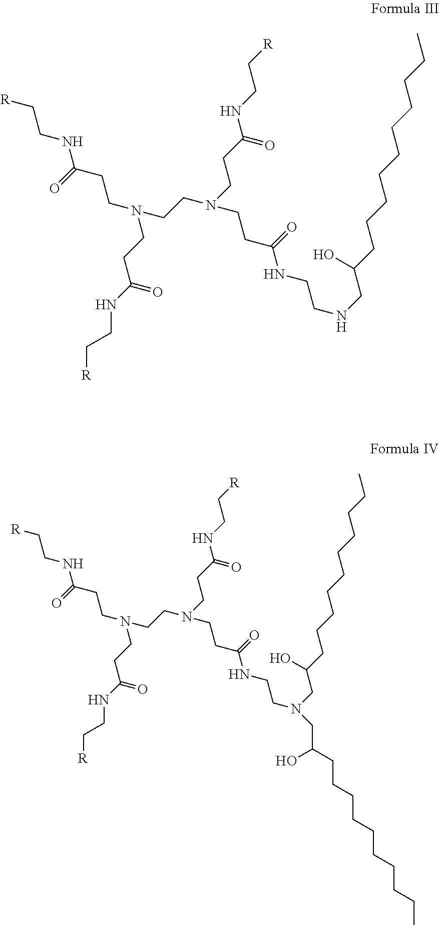

In any of Formulas II, III and IV, R may be a functional group that may or may not have reacted with additional hydrophobic units. For example, R may be OH, NH₂, SH, COOH, OR′, NHR′, SR′H, COOR′ or NR′₂, wherein R′ is the hydrophobic unit. The hydrophobic group R′ can include, for example, a linear or branched C₆-C₂₀ carbon chain which can have additional functional groups present.

Compositions. Compositions of the present invention are formed by mixing a keratin-peptide with a dendrimer. The resultant keratin-peptide/dendrimer compositions can exist in a variety of forms and combination of forms. For example, when mixing keratin-peptide and dendrimer, functional groups of the two components may interact with one another. This interaction may be, for example, chemical bonding or other associations of the functional groups. Chemical bonding may include covalent bonds, ionic bonds and hydrogen bonds. Association of the keratin-peptide and dendrimer can be through van der Waals interactions, dipole-dipole interactions, or other intermolecular interactions. Furthermore, a dendrimer may interact with one or more of keratin-peptide units and result in a keratin-peptide/dendrimer composition with a mixture of keratin-peptide/dendrimer complexes as explained below.

Dendrimers may include a functional group that can react or associate with a complementary functional group on the keratin-peptide. A “complementary functional group” is a moiety that can react or associate with a functional group on another molecule. Examples of functional groups and complementary functional groups that may undergo association include, for example, carboxylic acids and alcohols (to form esters), carboxylic acids and amines (to form amides), amino groups (NH₂) and leaving groups (e.g. tosylates, halides, mesylates, etc.) to form secondary amines, hydroxyl groups and leaving groups (e.g. tosylates, halides, mesylates, etc.) to form ethers, etc. Functional groups and complementary functional groups can also be polar groups (e.g., OH, SH, etc.) or groups capable of ionization (e.g., NH₂, COOH, etc.). In such embodiments association may occur by formation of ionic bonds, hydrogen bonds, and the like. Persons skilled in the art will recognize that there are numerous functional group/complementary functional group combinations that can be used to couple dendrimer with keratin-peptide.

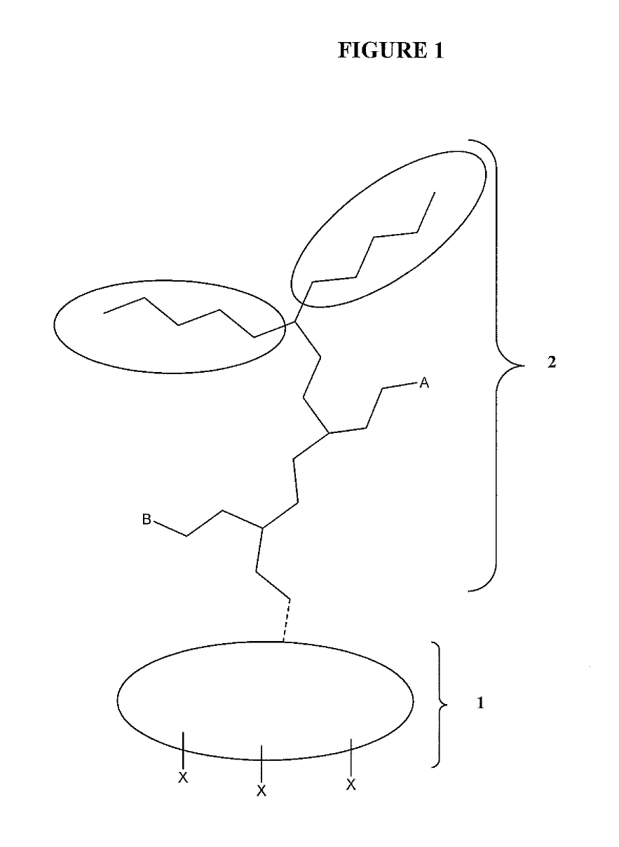

In some embodiments, the dendrimer interacts with the keratin-peptide in such a way that the keratin-peptide constituent remains at least partially exposed for interaction or react with keratin of a keratinaceous substrate. The keratin-peptide/dendrimer composition is shown schematically in FIG. 1 that includes a keratin-peptide constituent 1, in which X is a functional group on the keratin-peptide constituent, and a dendrimer portion 2, in which A and B represent sites available for interaction with additional keratin-peptide molecules, or for additional functionalization with a hydrophobic group. For example, A and B can independently be OH, NH₂, SH, COOH, OR′, NHR′, SR′H, COOR′ or NR′₂, where R′ is as defined above. Functional groups such as X on the keratin-peptide constituent 1 remain available for bonding with, associating with, coating, adhering to or attaching to keratin in the keratinaceous substrate. The dashed line in FIG. 1 is intended to denote an interaction between the dendrimer portion 2 and keratin-peptide 1. This interaction may be through, for example, a van der Waal's interaction, dipole-dipole interactions a hydrogen bond, an ionic bond or a covalent bond.

In some embodiments, a preformed dendrimer may have a reactive functional group that can be coupled to the keratin-peptide. Coupling can occur through a reactive functional group on the dendrimer and a complementary functional group of the keratin-peptide. The coupling of the keratin-peptide and the dendrimer may be through the mixing of the two, wherein the one or more of the functional groups on the dendrimer may associate with the keratin-peptide through interactions, such as van der Waal's interaction or hydrogen bonding. Alternatively, the preformed dendrimer can include or be reacted to include a group that interacts with and couples to a group on the keratin-peptide by forming a covalent bond. For example, oppositely charged groups on the dendrimer and the keratin-peptide can form an ionic bond. Either of these schemes would similarly yield the structure shown schematically in FIG. 1.

In other embodiments, one or more keratin-peptides may interact with the dendrimer without forming a chemical bond. As such, the keratin restorative composition may comprise of a mixture of unreacted keratin-peptide, unreacted dendrimer, single keratin-peptide/dendrimer complexes, secondary keratin-peptide/dendrimer complexes, and/or tertiary keratin-peptide/dendrimer complexes. As used herein, the term “complex” is to be interpreted to mean keratin-peptide and dendrimer that couple or associate to form a unit. For example, FIG. 1 is an example of a single keratin-peptide/dendrimer complex. A secondary keratin-peptide/dendrimer complex may be present when one dendrimer interacts with two keratin-peptides or two dendrimers interact with one keratin-peptide. A tertiary keratin-peptide/dendrimer complex may be present when a dendrimer interacts with three keratin-peptides or three dendrimers interact with one keratin-peptide.

The keratin-peptide/dendrimer composition comprising a dendrimer constituent and a keratin-peptide constituent offers several advantages as a treatment composition. For example, the keratin-peptide constituent remains available to react with keratin in the keratinaceous substrate, for example through a functional group such as X as shown in FIG. 1, to provide strengthening of the keratinaceous substrate similar to that achieved with the initial application of a hydrolyzed keratin. However, unlike the use of hydrolyzed keratin alone, the keratin-peptide/dendrimer composition in exemplary embodiments of the present invention also includes a dendrimer with a hydrophobic region. While the keratin-peptide constituent interacts or bonds with the nail keratin, the dendrimer portion is exposed to the environment. This can occur whether the keratin-peptide and dendrimer are mixed, associated, bonded, or chemically bonded. When this environment includes water, the dendrimer portion in essence forms a “shield” that prevents water from penetrating to the keratin-peptide constituent. Thus the keratin-peptide constituent is prevented from being solubilized by the water and the treatment composition remains intact for longer periods of time before additional treatment is required. In exemplary embodiments, the nail treatment remains intact for 2 or more days, 3 or more days, 4 or more days, 5 or more days, or 1 week or longer. When used as a nail treatment, the composition remains intact whether or not there is any additional coating placed on the nail over the nail treatment.

The keratin-peptide/dendrimer may be present in the formulation from about 0.01 to about 15% by weight. For example, keratin-peptide/dendrimer may be present in the formulation from about 0.1 to about 10% by weight, or from about 0.1 to about 5% by weight. The keratin-peptide and dendrimer may be present in a weight ratio from about 4:1 to about 1:60 based on the weight of keratin and dendrimer without solvent. For example, keratin-peptide and dendrimer may be present in a weight ratio of about 2:1, about 1:1, about 1:10, about 1:26, about 1:45, or about 1:52. In an embodiment, the keratin-peptide and dendrimer may be present in a weight ratio of about 2:1 or higher, about 1:1 or higher, about 1:10 or higher, about 1:26 or higher, about 1:45 or higher, or about 1:52 or higher. In an embodiment, the keratin-peptide and dendrimer may be present in a weight ratio of about 2:1 or lower, about 1:1 or lower, about 1:10 or lower, about 1:26 or lower, about 1:45 or lower, or about 1:52 or lower.

In some embodiments, the amount of keratin-peptide can range from about 0.0002-12% by weight, about 0.008-12% by weight. About 0.001-1.5% by weight, or about 0.002-0.0.25% by weight. For example, the keratin-peptide may be present in the formulation in an amount greater than about 0.0002% by weight, greater than about 0.002% by weight, greater than about 0.001% by weight, or greater than about 0.01% by weight. In an embodiment, the keratin-peptide may be present in the formulation in an amount less than about 12% by weight, less than about 10% by weight, less than about 7.5% by weight, less than about 4% by weight, less than about 2.5% by weight, less than about 1% by weight, less than about 0.5% by weight, or less than about 0.25% by weight.

In some embodiments, the amount of dendrimer can range from about 0.0002-14.75% by weight, about 0.002-3% by weight. About 0.01-13.5% by weight, or about 0.01-14.75% by weight. For example, the dendrimer may be present in the formulation in an amount greater than about 0.002% by weight, greater than about 0.02% by weight, or greater than about 0.01% by weight. In an embodiment, the dendrimer may be present in the formulation in an amount less than about 14.75% by weight, less than about 10% by weight, less than about 7.5% by weight, less than about 4% by weight, less than about 2.5% by weight, or less than about 1% by weight.

The present system allows the use of the keratin-peptide in an oil based carrier to penetrate, condition, and plasticize the nail. At the same time, the keratin-peptide/dendrimer composition binds to and seals the keratin substrate providing protection and improving resistance to common solvents such as acetone and water that are harmful to the structure of the keratin substrate. This reduces the severity of existing imperfections by providing additional bonding and protection as well as reducing the recovery time from those imperfections by arresting the propagation of cracks and weak spots between the cells in the keratin substrate.

Treatment compositions of the invention may further include one or more solvents. Solvents can be present in an amount up to about 99% of the composition, for example at about 90% of the composition. Typically, although not necessarily, solvents are non-polar or lipophilic solvents, including but not limited to ketones, alkyl acetates, alcohols, alkanes, alkenes, plant or nut oils and mixtures thereof. Specific non-limiting examples of solvents include acetone, ethyl acetate, butyl acetate, isopropyl alcohol, ethanol, methyl ethyl ketone, toluene, hexane, sweet almond oil, jojoba oil, rice bran oil, vitamin E acetate and mixtures thereof. The solvent used depends, in part, on the use of the composition and can be readily determined by persons skilled in the art.

Treatment compositions of the invention can include further additives such as an epoxy resin substantially composed of an ester fraction of an epoxidized esterified plant oil, or derivative thereof that provides adhesion when applied to a keratinaceous surface and cured. In such components the ester fraction includes one or more epoxidized fatty acid esters, or derivatives thereof. Each of the one or more fatty acid ester(s) is not a mono-, di- or triglyceride. The fatty acid ester(s), or derivative(s) thereof, can be an alkyl ester, such as a methyl ester. The resin can include an ester fraction of one or more additional epoxidized esterified plant oils, or derivatives thereof.

Treatment compositions of the invention can also include at least one polyhedral oligomeric silsesquioxane (POSS). Polyhedral oligomeric silsesquioxanes, as well as other components that can be used in compositions of the invention, are described in US Patent Application Publication No. 2014/0053859A1, which is incorporated herein by reference in its entirety.

Methods of Use.

Treatment composition and formulations according to the present invention can be used in a variety of products, particularly cosmetic products. The compositions can be rubbed, poured, sprinkled, or sprayed on, introduced into, or otherwise applied for cleansing, beautifying, promoting attractiveness, or altering appearance. Treatment composition and formulations of the invention are particularly suited for application to keratinaceous substrates, for example, skin, hair and nails. Compositions containing a keratin-peptide and dendrimer or keratin-peptide/dendrimer of the invention can be formulated into, for example, moisturizers, perfumes, lipsticks, eye and facial makeup preparations, cleansing shampoos, hair conditioners, permanent waves, hair colors, nail coatings, nail treatments and other cosmetic products or components of cosmetic products. Accordingly, treatment compositions according to the invention can include additional components satisfactory for use in these and other cosmetic compositions and products, so long as the additional components do not adversely affect the advantages of the present invention.

According to one aspect, the disclosed composition and formulation is a nail treatment or nail restorative composition for fingernails or toenails to prevent, treat, or ameliorate delamination, dryness, brittleness, cracking, chipping, splitting, texturing or other degradation of the nail material. According to an aspect, the disclosed compositions and formulations are applied to the exposed fingernails or toenails to treat the nail material to prevent nail brittleness, delamination, dryness, cracking, chipping, texturing or other imperfections. According to an aspect, the disclosed compositions and formulations are applied to the exposed fingernails or toenails to improve the appearance of the nail by binding together the structure and thus smoothing imperfections in the nail. According to an aspect, the disclosed compositions and formulations may be applied periodically from multiple times per day to once per week in a program for minimizing nail fracture or other damage.

Nail treatment compositions of the present invention may also serve as a decorative coating by being incorporated into a nail enamel, varnish, or artificial nail system. For example, in some embodiments the nail treatment composition may include a film forming polymer such as a cellulose ester. Non-limiting examples of cellulose esters include nitrocellulose and cellulose acetate alkylates such as cellulose acetate propionate and cellulose acetate butyrate and the like. In some embodiments, the nail treatment composition may also include a reactive (meth)acrylate that can be cured to form an acrylic coating. As known in the art, the term (meth)acrylate encompasses acrylates and/or methacrylates, including reactive monomers, and/or oligomers, and/or polymers. In some embodiments, the (meth)acrylates may be selected from the group consisting of acrylic acid; methacrylic acid; alkyl acrylates and methacrylates, such as methyl methacrylate, ethyl methacrylate, propyl methacrylate, methyl acrylate, ethyl acrylate, propyl acrylate, etc.; hydroxypropyl methacrylate (HPMA); hydroxyethyl methacrylate (HEMA, which may also serve as a solvent or co-solvent); THFMA; pyromellitic dianhydride di(meth)acrylate; pyromellitic dianhydride glyceryl dimethacrylate; pyromellitic dimethacrylate; methacroyloxyethyl maleate; 2-hydroxyethyl methacrylate/succinate; 1,3-glycerol dimethacrylate/succinate adduct; phthalic acid monoethyl methacrylate; acetoacetoxy ethyl methacrylate (AAEMA); and mixtures thereof. The nail treatment composition may include other components used in nail coatings as known in the art.

The nail treatment compositions of the present invention may further include a colorant. The colorant may be, for example a pigment or dye. In some embodiments the composition may comprise up to 10 wt % pigments and/or dyes.

The disclosed compositions may be applied to a nail using a medicine dropper or a brush. The compositions may be rubbed into the nail surface or may be left to penetrate or coat without mechanical assistance.

According to an aspect, the disclosed composition and formulation is a hair treatment or restorative composition for use on the hair to prevent, treat, or ameliorate dryness, brittleness, breaking or cracking or improve the texture or appearance of the hair. According to an aspect, the disclosed compositions and formulations are applied to the hair to prevent dryness, brittleness, breaking or cracking or to improve the texture or appearance of the hair. Compositions and formulations for application to hair include, but are not limited to, shampoos, conditioners, permanent waves, hair colors, hair sprays and hair treatments. Compositions and formulations of the invention can be applied to the hair by, for example, pouring, rubbing, spraying or brushing into the hair. Hair treatment or restorative composition and formulations of the invention can include other components used in hair products including, for example, colorants, fragrances, surfactants, emollients, emulsifiers, solvents, propellants, and the like. Persons skilled in the art will know of suitable components that are necessary or useful for the specific application.

According to an aspect, the disclosed composition and formulation is a skin treatment or restorative composition for use on the skin to prevent, treat, or ameliorate dryness, or cracking or to improve the texture or appearance of the skin. According to an aspect, the disclosed compositions and formulations are applied to the skin to prevent dryness or cracking, or to improve the texture or appearance of the skin. Compositions and formulations for application to skin include, but are not limited to moisturizers, perfumes, lipsticks, eye and facial makeup preparations, sprays and skin treatments. Compositions and formulations of the invention can be applied to the skin by, for example, pouring, rubbing, sprinkling, spraying or brushing onto the skin. Skin treatment or restorative composition and formulations of the invention can include other components used in skin products including, for example, colorants, fragrances, surfactants, emollients, emulsifiers, solvents, propellants, and the like. Persons skilled in the art will know of suitable components that are necessary or useful for the specific application.

### EXAMPLES

Aspects of the present invention demonstrated by the following examples that are demonstrative only and not intended to limit the invention.

### Example 1

Set forth below are exemplar ranges of components that may be included in keratin treatments according to the invention. The keratin treatments may be incorporated into a wide range of products including nail treatments (such as base coats, top coats, and color treatments) including curable nail treatments, enamels, artificial nails and the like. The keratin treatment may also be incorporated into hair products, such as conditioners, moisturizers, shampoos, gels, mousse, etc. The keratin treatment may also be incorporated into topical treatments such as lotions, moisturizers, skin creams and treatments, make-up, lipstick, etc. Based on the present disclosure and the exemplary ingredients listed below, persons skilled in the art will recognize uses and quantities applicable to particular uses.

### Example 2

A hydrophobic dendrimer was prepared from Polyamidoamine (PAMAM) and an epoxidized hydrophobic precursor in isopropanol. The reaction product was analyzed using High Performance Liquid Chromatography (HPLC) and Matrix Assisted Laser Desorption/Ionization—Time of Flight Mass Spectroscopy (MALDI-TOF). The MALDI-TOF data reflect a typical distribution that occurs during the hydrophobe reaction which uses epoxide chemistry. The presence of the parent mass peak, and the Na+ and K+ adducts commonly seen by this technique are reflected as “triplet” peaks. It is important to note that because of the non-linear detection inherent in this technique, the relative heights of the various species do not necessarily reflect the actual amount present in the sample.

The components in the HPLC were assumed to elute in the order of hydrophobicity, and comparisons with MALDI-TOF permit general assignment of major components relating to their hydrophobe content. The earliest-eluting peak is suspected to be unreacted component at, and the following major peaks are assumed to be the mono, di, and tri-epoxylated adducts. The MALDI-TOF results indicate the presence of even higher epoxylated species which apparently do not show up in the HPLC method and are expected to be minor components in any case.

Presuming that the above assumptions are correct, and based on the further assumption that HPLC peak areas represent molar areas allows an estimate of the relative amounts of the different species present. These estimates are shown below.

| Component | Mole % | Weight % |
|---|---|---|
| Unreacted 100% OH | 42.0 | 32.7 |
| Monoepoxy | 23.4 | 24.2 |
| Diepoxy | 22.2 | 29.2 |
| Triepoxy | 8.7 | 13.9 |

The embodiments illustrated and discussed in this specification are intended only to teach those skilled in the art the best way known to the inventors to make and use the invention. Nothing in this specification should be considered as limiting the scope of the present invention. All examples presented are representative and non-limiting. The above-described embodiments of the invention may be modified or varied, without departing from the invention, as appreciated by those skilled in the art in light of the above teachings. It is therefore to be understood that, within the scope of the claims and their equivalents, the invention may be practiced otherwise than as specifically described.

We claim:

1. A keratin restorative composition comprising:
a keratin-peptide; and
a dendrimer.

2. The keratin restorative composition according to claim 1, wherein a weight ratio of keratin-peptide to dendrimer is from about 4:1 to about 1:60.

3. The keratin restorative composition according to claim 1, wherein said keratin-peptide comprises a partially-hydrolyzed keratin.

4. The keratin restorative composition according to claim 1, comprising a mixture of the keratin-peptide and the dendrimer.

5. The keratin restorative composition according to claim 1, wherein the keratin-peptide and the dendrimer are associated.

6. The keratin restorative composition of claim 5, wherein the keratin-peptide and the dendrimer are associated by an interaction selected from the group consisting of van der Waal's interaction, dipole-dipole interaction, covalent bonding, ionic bonding and hydrogen bonding.

7. The keratin restorative composition according to claim 1, wherein the dendrimer comprises a functional group that interacts react with the keratin-peptide.

8. The keratin restorative composition according to claim 1, wherein the dendrimer comprises a functional group selected from the group consisting of OH, NH₂, SH, and COOH.

9. The keratin restorative composition according to claim 1, wherein the keratin-peptide remains at least partially exposed for interaction or reaction with keratin of a keratinaceous substrate and the dendrimer functional group extends unidirectionally from the keratin-peptide constituent.

10. The keratin restorative composition according to claim 1, wherein the dendrimer comprises at least one hydrophobic group.

11. The keratin restorative composition according to claim 10, wherein the hydrophobic group includes a linear or branched C₆-C₂₀ carbon chain optionally substituted with one or more functional groups.

12. The keratin restorative composition according to claim 10, wherein the dendrimer comprises a compound having the structure 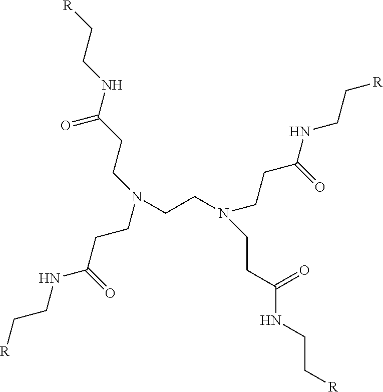
wherein R is selected from the group consisting of OH, NH₂, SH, COOH, OR′, NHR′, SR′H, COOR′ and NR′₂;
R′ is a hydrophobic group; and
the dendrimer contains at least one hydrophobic group.

13. The keratin restorative composition according to claim 1, further comprising a solvent.

14. The keratin restorative composition according to claim 1, further comprising one or more components selected from the group consisting of a film forming polymer, a reactive (meth)acrylate, an epoxidized esterified plant oil, a polyhedral oligomeric silsesquioxane, and a colorant.

15. A method for ameliorating a condition of a keratinaceous substrate comprising:
applying to the keratinaceous substrate a keratin restorative composition comprising:
a keratin-peptide; and
a dendrimer.

16. The method of claim 15, wherein the condition is selected from the group consisting of nail delamination, brittleness, cracking, chipping, splitting, dryness, and texturing.

17. The method of claim 15, wherein the keratinaceous substrate is selected from skin, nails and hair.

18. A method of making a keratin restorative composition comprising mixing a keratin-peptide and a dendrimer.

19. The method of claim 18, wherein mixing comprises chemically reacting.

20. The method of claim 18, wherein mixing comprises forming a covalent, ionic, or hydrogen bond.
